(19) United States
(12) Patent Application Publication
(10) Pub. No.: US 2023/0204156 A1
(43) Pub. Date: Jun. 29, 2023

(54) METHOD, SYSTEM AND MEDIUM FOR LUBRICATION ASSESSMENT

(71) Applicant: AKTIEBOLAGET SKF, Goteborg (SE)

(72) Inventors: Kaihuan ZHANG, Hangzhou (CN); Gang CHENG, Shanghai (CN)

(21) Appl. No.: 17/986,216

(22) Filed: Nov. 14, 2022

(30) Foreign Application Priority Data
Dec. 23, 2021 (CN) 202111588573.7

(51) Int. Cl. F16N 29/00

(52) U.S. Cl. CPC F16N 29/00

(57) ABSTRACT

A method, system and non-transient computer-readable storage medium for lubrication assessment. The method includes acquiring working condition data related to target lubrication, condition monitoring data related to the target lubrication, and lubrication assessment data related to the target lubrication; preprocessing the acquired working condition data, condition monitoring data, and lubrication assessment data; performing data integration on the preprocessed working condition data, condition monitoring data, and lubrication assessment data to obtain an integrated data set; performing feature extraction on data in the integrated data set according to data types and data characteristics based on the integrated data set to obtain a feature data set related to the target lubrication; establishing a lubrication analysis model for assessment of the target lubrication based on the feature data set related to the target lubrication; and assessing the target lubrication and generating a lubrication assessment result, based on the lubrication analysis model.

### CROSS-REFERENCE

This application claims priority to Chinese Patent Application No. 202111588573.7 filed on Dec. 23, 2021, the contents of which are fully incorporated herein by reference.

### TECHNOLOGICAL FIELD

The present disclosure relates to a field of lubrication management, and more particularly to a method, system and medium for lubrication assessment.

### BACKGROUND

Lubrication management runs through an entire life cycle of an equipment, so it is necessary to detect and assess a lubrication status of the equipment. At present, lubrication detection and assessment mainly focus on two aspects. On the one hand, it focuses on physicochemical properties such as composition, viscosity, consistency and contamination of lubricant. On the other hand, it focuses on field applications. Detection and analysis of the physicochemical properties of a lubricant always relies on sampling the lubricant and sending the sampled lubricant to a laboratory for an analysis, such as a content analysis, an infrared thermography analysis and an oil analysis in the laboratory. For field applications, a regular field inspection is required by using special equipment. Both methods are invasive (e.g., equipment may need to be disassembled to find lubrication points for the equipment and sample lubricant at the lubrication points) and are off-line for analysis (e.g., the sampled lubricant needs to be sent to the laboratory) and therefore are not timely.

Although some online lubrication detection and assessment methods have recently emerged, such as an oil film analysis, an infrared thermograph analysis, and an even ultrasonic analysis, these methods usually require special and complex equipment and systems that are difficult to deploy in field. In addition, most of these methods are only aimed at a single lubrication indicator, which is insufficient to reflect the overall and comprehensive status of the equipment lubrication.

Therefore, it is necessary to develop a non-invasive, timely, comprehensive and multi-dimensional lubrication detection and assessment technology.

### SUMMARY OF THE DISCLOSURE

According to an aspect of the present disclosure, a lubrication assessment method is provided. The method comprises: acquiring working condition data related to target lubrication, condition monitoring data related to the target lubrication, and lubrication assessment data related to the target lubrication; preprocessing the acquired working condition data, condition monitoring data, and lubrication assessment data to obtain preprocessed working condition data, preprocessed condition monitoring data, and preprocessed lubrication assessment data; performing data integration on the preprocessed working condition data, the preprocessed condition monitoring data, and the preprocessed lubrication assessment data to obtain an integrated data set; performing feature extraction on data in the integrated data set according to data types and data characteristics based on the integrated data set to obtain a feature data set related to the target lubrication; establishing a lubrication analysis model for assessment of the target lubrication based on the feature data set related to the target lubrication; and assessing the target lubrication and generating a lubrication assessment result, based on the lubrication analysis model.

In some embodiments, performing feature extraction on data in the integrated data set according to data types and data characteristics based on the integrated data set to obtain the feature data set related to the target lubrication may include: extracting features of the working condition data based on the integrated data set to obtain working condition features; extracting features of the condition monitoring data based on the integrated data set to obtain condition monitoring features; extracting features of the lubrication assessment data based on the integrated data set to obtain lubrication assessment features; obtaining the feature data set related to the target lubrication based on the working condition features, the condition monitoring features, and the lubrication assessment features.

In some embodiments, obtaining the feature data set related to the target lubrication based on the working condition features, the condition monitoring features, and the lubrication assessment features may include: obtaining fused feature data by feature fusion processing based on the working condition features, the condition monitoring features, and the lubrication assessment features, and generating the feature data set related to the target lubrication based on the fused feature data.

In some embodiments, establishing the lubrication analysis model for assessment of the target lubrication based on the feature data set related to the target lubrication may include: establishing a lubrication anomaly detection model for detecting lubrication anomaly based on the feature data set related to lubrication; establishing a lubrication failure mode classification model for classifying lubrication failure modes based on the feature data set related to lubrication; establishing a lubrication level classification model for classifying lubrication levels based on the feature data set related to lubrication; and establishing a lubricating indicator prediction model for predicting lubricating indicators based on the feature data set related to lubrication.

In some embodiments, assessing lubrication and generating a lubrication assessment result based on the lubrication analysis model includes: detecting lubrication anomaly and generating a lubrication anomaly detection result based on outputs of the lubrication anomaly detection model; classifying lubrication failure modes and generating a lubrication failure mode classification result based on outputs of the lubrication failure mode classification model; classifying lubrication levels and generating a lubrication level classification result based on outputs of the lubrication level classification model; predicting lubrication indicators and generating a lubrication indicator prediction result based on the lubrication indicator prediction model; and generating a lubrication health assessment result based on at least one of the lubrication anomaly detection result, the lubrication failure mode classification result, the lubrication level classification result, and the lubrication indicator prediction result.

In some embodiments, the preprocessing the acquired working condition data, condition monitoring data, and lubrication assessment data includes performing at least one of a data deduplication processing, a data denoising processing, a data encoding processing and a data filtering processing.

In some embodiments, performing data integration on the preprocessed working condition data, the preprocessed condition monitoring data, and the preprocessed lubrication assessment data includes: performing at least one of synchronization, alignment, and correction processing on the preprocessed working condition data, the preprocessed condition monitoring data, and the preprocessed lubrication assessment data.

In some embodiments, the method further comprises optimizing the target lubrication based on the lubrication assessment result.

According to another aspect of the present disclosure, a lubrication assessment system is provided. The system comprises a data collector and a processor connected to the data collector. The data collector may be configured to acquire working condition data related to target lubrication, condition monitoring data related to the target lubrication, and lubrication assessment data related to the target lubrication. The processor may be configured to: preprocess the acquired working condition data, condition monitoring data, and lubrication assessment data to obtain preprocessed working condition data, preprocessed condition monitoring data, and preprocessed lubrication assessment data; perform data integration on the preprocessed working condition data, the preprocessed condition monitoring data, and the preprocessed lubrication assessment data to obtain an integrated data set; perform feature extraction on data in the integrated data set according to data types and data characteristics based on the integrated data set to obtain a feature data set related to the target lubrication; establish a lubrication analysis model for assessment of the target lubrication based on the feature data set related to the target lubrication; and assess the target lubrication and generate a lubrication assessment result based on the lubrication analysis model.

According to another aspect of the present disclosure, a non-transient computer-readable storage medium having instructions stored thereon is provided, and the instructions are executed by a computer to perform the above lubrication assessment method.

The lubrication assessment method, system and computer-readable medium of the present disclosure can realize utilization and fusion of multi-signal, multi-working condition, and multi-dimensional data related to lubrication, extract more comprehensive features related to lubrication, and therefore can establish more effective correlation models between targets (or process statuses) to which lubrication is applied and lubrication statuses, thereby obtaining more sensitive and accurate indicators, to finally reflect and assess various conditions and statuses about lubrication online and quantitatively.

Furthermore, the lubrication assessment method, system, and non-transient computer-readable storage medium of the present disclosure can enrich and enhance existing lubrication inspection and assessment methods from a non-invasive, timely, and quantitative perspective. With the method, system and non-transient computer-readable storage medium of the present disclosure, lubrication problems can be discovered in time, lubrication failure modes and severity can be classified and graded online, lubrication performance can be predicted in advance, and process parameters can be optimized in real time. In addition, with the method, system and non-transient computer-readable storage medium of the present disclosure, more objective, quantitative, and timely indicators can be obtained for assessment and control of lubrication performance, and even more comprehensive lubrication performance standards can be output.

Furthermore, by leverage of the method, system and non-transient computer-readable storage medium of the present disclosure, it is possible to monitor, assess, control and optimize lubrication process, status and performance in a continuous closed loop, thereby greatly enhancing lubrication assessment, control and solution capabilities. By processing and modeling acquired data based on large data or machine learning, it is possible to support assessment and optimization of lubrication in a digitalized and intelligent way.

### BRIEF DESCRIPTION OF THE DRAWINGS

The system can be better understood with reference to the following description in conjunction with accompanying drawings. Components in the drawings are not to scale, emphasis instead being placed upon illustrating principles of the present disclosure. Furthermore, in the drawings, like or identical reference numerals represent like or identical elements.

FIG. 1 is a flow chart illustrating a method for lubrication assessment according to one or more embodiments of the present disclosure.

FIG. 2 is a modular framework diagram illustrating a method or system for lubrication assessment, decision making and optimization according to one or more embodiments of the present disclosure.

FIGS. 3A-3D are an exemplary lubrication anomaly degree trend diagrams according to the method and system of the present disclosure, which includes anomaly degree trend diagrams of four lubrication anomaly indicators.

FIG. 4 is a confusion matrix of lubrication failure mode classification results in a lubrication assessment process during testing of the method and system of the present disclosure.

FIG. 5 is a confusion matrix of lubrication level classification results in a lubrication assessment process during testing of the method and system of the present disclosure.

FIG. 6 is a chart illustrating predicted results of a lubrication indicator in a lubrication assessment process during testing of the method and system of the present disclosure.

FIG. 7 is a schematic diagram of fitting and regression performance of the lubrication indicator prediction of FIG. 6.

FIG. 8 is a radar chart of an exemplary lubrication health assessment based on partial health assessment results obtained using the lubrication assessment method and system of the present disclosure.

### DETAILED DESCRIPTION

It should be understood that the following description of embodiments is given for purposes of illustration only and not limitation. Exemplary division of functional blocks, modules or units shown in the drawings should not be construed as implying that these functional blocks, modules or units must be implemented as physically separate units. The functional blocks, modules or units shown or described may be implemented as separate units, circuits, chips, functions, modules or circuit elements. One or more functional blocks or units may also be implemented in a common circuit, chip, circuit element or unit.

Although the present disclosure makes various references to certain modules in a system according to an embodiment of the present disclosure, any number of different modules may be used and run on user terminals and/or servers. The modules are illustrative only, and different aspects of the system and method may use different modules.

Flow charts are used in the present disclosure to illustrate operations performed by a system according to one or more embodiments of the present disclosure. It should be understood that preceding or following operations are not necessarily performed in exact order. Rather, various steps may be processed in reverse order or concurrently, as desired. At the same time, other operations may be added to these procedures, or a step or steps may be removed from these procedures.

It should be understood that an object to be lubricated in the present disclosure may be any mechanical system, mechanical equipment, or mechanical component and so on, which requires lubrication. In addition, the lubrication may also be associated with a location where the lubrication is performed (i.e., the location of the lubrication point). The location of the lubrication point may be selected according to actual situations of the actual mechanical system, mechanical equipment, mechanical component or actual requirements. The embodiments of the present disclosure are not limited by specific lubrication objects or locations where lubrication is performed. Lubrication assessment obtained by the lubrication assessment method of the present disclosure may be assessment for lubrication status of any lubrication object, such as a mechanical system, mechanical equipment, mechanical component, and the like, and may also be assessment for lubrication status at any location of any lubrication object where lubrication is performed. Target lubrication in the present disclosure refers to the lubrication that needs to be assessed, which may include lubrication for any mechanical system, mechanical equipment, and mechanical component, as well as lubrication for each lubrication point within these lubrication objects.

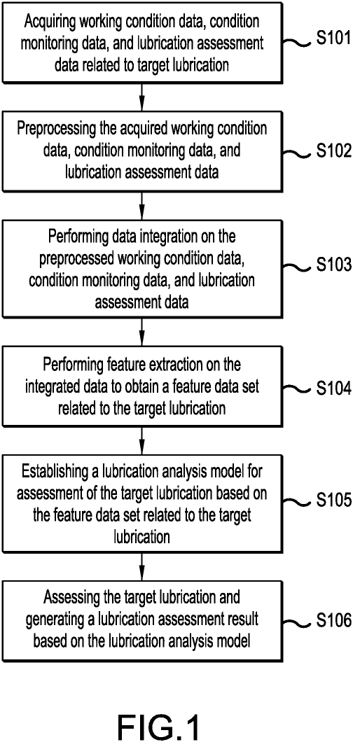

FIG. 1 schematically illustrates a flow chart of a method for lubrication assessment according to one or more embodiments of an aspect of the present disclosure.

Referring to FIG. 1, at S101, working condition data related to target lubrication, condition monitoring data related to the target lubrication, and lubrication assessment data related to the target lubrication are collected.

The working condition data may mainly include data reflecting a real-time working condition of a lubricating object, which may significantly influence the lubrication. For example, the working condition data may include data related to the following: a component rotational speed at the lubrication point, a component load at the lubrication point, an ambient temperature at the lubrication point, an ambient humidity at the lubrication point, an ambient particle concentration at the lubrication point, a type of a component at the lubrication point, parameters of the component at the lubrication point, a lubrication type, a friction type, and the like. It should be appreciated that the embodiments of the present disclosure are not limited by the specific contents and types of the above-mentioned working condition data. In practical applications, the working condition data to be acquired may also be determined according to actual requirements and actual application scenarios.

The condition monitoring data may be data reflecting a condition at the target lubrication point. For example, the condition monitoring data may include data related to a vibration, a temperature, etc. at the target lubrication point. More specifically, the condition monitoring data may include data related to the lubrication point on the following: a real-time vibration, a temperature, a ferrous particle content, a moisture content, and the like. It should be appreciated that the embodiments of the present disclosure are not limited by the specific contents and types of the above-mentioned condition monitoring data. In practical applications, the condition monitoring data to be acquired may also be determined according to actual requirements and actual application scenarios.

The lubrication assessment data may be historical data on lubrication obtained by lubrication sampling and inspection. For example, lubrication assessment data may be acquired and recorded by manual logging, sampling inspection and laboratory analysis. For example, the lubrication assessment data may include data on: lubrication status, lubrication quantity, lubricant quality, lubricant cleanliness, lubricant viscosity, lubricant layer thickness, cleanliness, surface finish, and the like.

In addition, specific sources and acquisition methods of the lubrication-related working condition data, condition monitoring data, and lubrication assessment data in the present disclosure may be diverse. For example, the target working condition data related to lubrication may be directly obtained from a control system, working system or other externally connected systems or servers (such as a data acquisition and monitoring system) for the lubrication object according to a predetermined sampling frequency, or may also be obtained from other sources or by other means. For example, the condition monitoring data related to lubrication may be acquired in a predetermined sampling frequency from various sensors disposed on the lubrication object, or around the lubrication object, or around the lubrication point. For example, historical data for lubrication assessment may be collected from the control system, working system, or other externally connected systems. The lubrication assessment data may also be obtained by manual logging, sampling inspection and laboratory analysis according to actual requirements, and the obtained assessment data may be used as historical data on lubrication assessment. It should be appreciated that the lubrication-related working condition data, condition monitoring data, and lubrication assessment data of the present disclosure may also be obtained by other means. The embodiments of the present disclosure are not limited by their specific sources and how they are obtained.

At S102, the acquired working condition data, condition monitoring data, and lubrication assessment data may be preprocessed to obtain preprocessed working condition data, preprocessed condition monitoring data, and preprocessed lubrication assessment data.

The process of preprocessing the working condition data, condition monitoring data and lubrication assessment data acquired above may include processing the data using various algorithms according to characteristics of the data, so as to select currently required valid data, reduce and suppress invalid data and improve data quality. In some embodiments, preprocessing the working condition data, the condition monitoring data, and the lubrication assessment data may include performing, according to characteristics of the data, at least one of a data deduplication processing, a data denoising processing, a data encoding processing and a data filtering processing.

The data deduplication processing is intended to remove duplicate data. For example, duplicate data may be retrieved and removed based on data such as timestamps, process numbers, and the like.

The data denoising processing is intended to remove outliers in data and realize optimization of data. For example, methods such as distance-based detection, statistics-based detection, distribution-based outlier detection, density clustering detection, boxplot detection and the like may be used to perform denoising on signal data to remove outliers in the data.

The data encoding processing is intended to use different encoding schemes to process data formats as required, so as to obtain encoded data. For example, a required target data format may be determined according to modeling, analysis and assessment, and the data may be encoded accordingly based on the target data format to facilitate subsequent processing.

The data filtering processing is intended to identify and remove noise in data and improve a contrast of valid feature information in data. For example, data filtering may be implemented by using a weighted average filter, a median filter, a Gaussian filter, a Wiener filters, and other methods.

The above only exemplifies several specific processing methods that the preprocessing may include. It should be appreciated that other preprocessing methods may also be selected according to actual requirements. In addition, according to characteristics of data, one or more of the above-mentioned preprocessing methods may be selected to perform preprocessing of data.

In an example of preprocessing of the working condition data, for data on a component rotational speed at the lubrication point, a component load at the lubrication point, ambient temperature at the lubrication point, ambient humidity at the lubrication point, ambient particle concentration at the lubrication point, outliers may be filtered out using dynamic boxplot. Data on a type of the component at the lubrication point, parameters of the component at the lubrication point, a lubrication type, and a friction type are all disordered discrete variables, so dummy variable discretized encoding method may be performed on these data.

In an example of preprocessing of the condition monitoring data, for vibration data, its envelope spectrum in high frequency band is taken; and for temperature, iron and water content data, smooth filtering is performed.

In an example of preprocessing of the condition monitoring data, for discrete data in the lubrication assessment data, dummy variable encoding is performed; and for continuous data, outliers may be extracted using the 3-sigma principle.

For example, the preprocessed working condition data, the preprocessed condition monitoring data, and the preprocessed lubrication assessment data may be respectively recorded as different data subsets according to the above data categories, for example, as data subsets D_{cond}, D_{como}, D_{lub}.

At S103, the data integration may be performed on the preprocessed working condition data, the preprocessed condition monitoring data, and the preprocessed lubrication assessment data to obtain an integrated data set.

In some embodiments, the performing data integration on the preprocessed working condition data, the preprocessed condition monitoring data, and the preprocessed lubrication assessment data to obtain an integrated data set may include: performing at least one of synchronization, alignment, and data correction processing on the preprocessed working condition data, the preprocessed condition monitoring data, and the preprocessed lubrication assessment historical data.

For example, the synchronization, alignment and correction of the preprocessed multi-source data D_{cond}, D_{como}, D_{lub}, may be completed by using a plurality of algorithms such as interpolation and translation algorithms based on a standard clock source, and then a complete data set D for lubrication monitoring, assessment and optimization may be obtained.

Specifically, for example, when the working condition data, condition monitoring data and lubrication assessment data related to the target lubrication are acquired by periodic sampling, due to different sampling frequencies selected and different starting times of the sampling process, the acquired working conditions data, condition monitoring data and lubrication assessment data, for example, have different starting points in the time axis, and their respective durations are different. It is also possible that some data are missing or significantly inaccurate due to anomalies in the sampling process, resulting in the problem of the acquired original data having incomplete data content in the spatial dimension, and being discontinuous and having inconsistent time series in the time dimension. In this case, for example, the data may be processed in the time dimension based on a standard clock source, so as to realize synchronization and alignment between multi-source data. At the same time, various algorithms such as the interpolation algorithm and the translation algorithm may be used to correct and complete data values (i.e., processing in the spatial dimension), so as to obtain the integrated complete data set for lubrication monitoring and assessment.

At S104, the feature extraction is performed on data in the integrated data set according to data types and data characteristics based on the integrated data set to obtain the feature data set related to the target lubrication.

In some embodiments, the performing of feature extraction may include: extracting features of the working condition data based on the integrated data set to obtain working condition features; extracting features of the condition monitoring data based on the integrated data set to obtain condition monitoring features; extracting features of the lubrication assessment historical data based on the integrated data set to obtain lubrication assessment features; and obtaining the feature data set related to the lubrication based on the working condition features, the condition monitoring features, and the lubrication assessment features.

The following extraction processes may be performed in any order or in parallel: extracting features of the working condition data in the data set, extracting features of the condition monitoring data in the data set, and extracting features of the lubrication assessment historical data in the data set. In addition, the above feature extraction processes may adopt a plurality of feature extraction methods, and each extraction process may adopt the same extraction method or different extraction methods. An exemplary feature extraction method for the above-described feature extraction processes is illustrated below. For example, depending on actual requirements, the data extraction for the data set may include, for example, time domain feature extraction, frequency feature extraction, time-frequency domain feature extraction, waveform feature extraction, and the like.

The time-domain feature extraction refers to extracting time-domain features of data (such as collected signals), including but not limited to mean, variance, standard deviation, maximum value, minimum value, root mean square, peak-to-peak value, skewness, kurtosis, waveform index, pulse index, margin index, and the like. Frequency feature extraction refers to extracting frequency features of data, including but not limited to mean square frequency, frequency variance, frequency band energy, and the like. Time-frequency domain feature extraction refers to extracting time-frequency domain features of data, including but not limited to frequency band energy or time domain characteristics of signals after wavelet decomposition or empirical mode decomposition. Waveform feature extraction refers to extracting waveform features of data, which, for example, when the data is a collected signal, includes but is not limited to an area enclosed by the signal waveform, maximum/minimum derivatives, rising edges, falling edges, and the like.

For example, in an example process for working condition feature extraction, for discrete working condition data, dummy variable encoding is used directly as a feature; for continuous working condition data, an average of a sliding window is used as a feature.

In an example process for condition monitoring feature extraction, for vibration data, a total value of its envelope spectrum within a certain range is taken as a vibration feature, and for temperature and content data, its sliding average value is taken as a temperature feature.

In an example process for lubrication assessment feature extraction, for discrete assessment data, dummy variable encoding is used directly as a feature; for continuous assessment data, an average of the sliding window is used as a feature.

In some examples, while performing the feature extraction, standardization processing is performed on feature data in each dimension, that is, an average value in the dimension is subtracted from an original feature value, and then divided by a standard deviation of the dimension.

As for formation of the feature data set, for example, the feature data set may be formed by directly including the working condition features, the condition monitoring features, and the lubrication assessment features. Alternatively, the working condition features, the condition monitoring features, and the lubrication assessment features may be further processed, and the feature data set is obtained based on processing results. The embodiments of the present disclosure are not limited by a specific generation method and content of the feature data set.

In some embodiments, the obtaining the feature data set related to the target lubrication based on the working condition features, the condition monitoring features, and the lubrication assessment features may further include: obtaining fused feature data by feature fusion processing based on the working condition features, the condition monitoring features, and the lubrication assessment features, and generating the feature data set based on the fused feature data.

For example, based on the feature data set obtained by extraction, feature fusion of different levels and dimensions may be performed to obtain an effective multi-dimensional feature vector. For example, the multi-dimensional feature vector may be expressed as F = {Fᵢ|i = 1,2, ... , k}, where k represents a dimension after feature extraction and fusion. F includes fused features of the working condition features, condition monitoring features and lubrication assessment features. The extracted and fused features herein may be features used for lubrication assessment, are sensitive to lubrication status, and are not easily affected by disturbances caused by working conditions.

For example, the method of feature layer deep fusion may be used, that is, fusing the extracted original features (e.g., the working condition features, the condition monitoring features, the lubrication assessment features) in different dimensions based on distance algorithm, similarity algorithm, weighted average algorithm, principal component analysis algorithm, etc., and obtaining fused features that integrate original feature information from the feature depth direction. For example, a method of feature fusion at different levels such as a signal level, a working status level and the like may also be adopted to obtain fused features integrating the original feature information from the feature width direction. It should be appreciated that the above only provides exemplary fusion methods, and different data fusion methods may be adopted according to actual requirements. The embodiments of the present disclosure are not limited by a specific method of data fusion.

Based on the above, according to actual requirements, by extracting the working condition features, condition monitoring features and lubrication assessment features related to lubrication from the integrated data set using various feature extraction methods, and obtaining the target feature data set based on the working condition features, condition monitoring features and lubrication assessment features, the obtained target feature data set can comprehensively reflect characteristics of lubrication in multiple aspects. Compared with existing technical solutions that only extract a single feature and perform only a single feature extraction method, the feature data set related to lubrication obtained by the feature extraction method of the present disclosure can more comprehensively reflect characteristics of lubrication in multiple levels, multiple dimensions, and multiple aspects, which are conducive to subsequent realization of a more comprehensive and accurate assessment of lubrication based on the feature data set.

At S105, a lubrication analysis model is established for assessment of the target lubrication based on the feature data set related to the target lubrication.

Based on the feature data set related to lubrication obtained from S104, different types of analysis and assessment models may be constructed for various lubrication assessment applications using comprehensive methods and algorithms. It should be appreciated that establishment of various analysis and assessment models related to lubrication described in the following embodiments of the present disclosure is only intended to be an example, rather than a specific limitation to the analysis and assessment models. Analysis and assessment models of other aspects related to lubrication may be established and configured according to actual requirements, and each model may also be selected and configured according to characteristics of applications.

For example, in some embodiments, the establishing the lubrication analysis model for assessment of the target lubrication based on the feature data set related to lubrication may include establishing a lubrication anomaly detection model for detecting lubrication anomaly based on the feature data set related to lubrication. The lubrication anomaly detection model may use a variety of anomaly detection methods to detect an anomaly risk of the target lubrication and relevant lubrication performance. The anomaly detection methods may include, but are not limited to, a K-sigma method, a boxplot method, KNN, LOF, one-class-SVM, etc., for example.

For example, in some embodiments, the establishing the lubrication analysis model for assessment of the target lubrication based on the feature data set related to lubrication may include establishing a lubrication failure mode classification model for classifying lubrication failure modes based on the feature data set related to lubrication. The lubrication failure mode classification model may use a variety of classification methods to detect and classify modes of the target lubrication statuses and relevant lubrication performance. The classification methods used therein may include, but are not limited to, Logistic regression, Bayesian, SVM, KNN, Decision tree, Random forest, XGBoost, etc., for example.

For example, in some embodiments, the establishing the lubrication analysis model for assessment of the target lubrication based on the feature data set related to lubrication may include: establishing a lubrication level classification model for classifying lubrication levels based on the feature data set related to lubrication. The lubrication level classification model may use a variety of classification methods to detect and classify levels of the target lubrication and relevant lubrication performance. The classification methods used therein may include, but are not limited to, Logistic regression, Bayesian, SVM, KNN, Decision tree, Random forest, XGBoost, etc., for example.

For example, in some embodiments, establishing the lubrication analysis model for assessment of the target lubrication based on the feature data set related to lubrication may include: establishing a lubricating indicator prediction model for predicting lubricating indicators based on the feature data set related to lubrication. The lubricating indicator prediction model may use a variety of correlation analysis and regression methods to fit and predict lubrication indicators of the target application and trends of relevant performance. The correlation analysis and regression methods used therein include, but are not limited to, Linear regression, Ridge regression, Lasso regression, SVR, Random forest, etc., for example.

Establishment and training of the above-described exemplary lubrication-related analysis models are based on the feature data set related to lubrication. In conjunction with the foregoing description of the present disclosure, it can be known that the feature data set related to lubrication is obtained based on feature extraction of the working condition data, condition monitoring data, and lubrication assessment historical data related to lubrication. By combining the lubrication assessment historical data, feature data may be labeled. The labeled feature data are used as an input of a corresponding analysis model, so that each analysis model can be effectively trained. Since the feature data set related to lubrication obtained by the aforementioned feature extraction method of the present disclosure can more comprehensively reflect characteristics of lubrication at multiple levels, multiple dimensions, and multiple aspects, outputs of analysis models established and trained based on the feature set will be able to reflect more comprehensive and accurate status about the lubrication, and thus is further beneficial to realize a more comprehensive and accurate assessment of lubrication status based on the outputs of the analysis models subsequently. It should be appreciated that the lubrication assessment historical data may only be used in the process of establishing and training the analysis and assessment model of the present disclosure. In the process of using the analysis and assessment model after training, it is no longer necessary to collect lubrication assessment historical data.

At S106, the lubrication is assessed and a lubrication health assessment result is generated, based on the lubrication analysis model.

For example, in some embodiments, based on outputs of the lubrication anomaly detection model, anomalies of lubrication may be detected and the lubrication anomaly detection result may be generated. For example, in some embodiments, based on outputs of the lubrication failure mode classification model, failure modes of lubrication may be classified and the lubrication failure mode classification result may be generated. For example, in some embodiments, based on outputs of the lubrication level classification model, levels of lubrication may be classified and the lubrication level classification result may be generated. For example, in some embodiments, based on the lubrication indicator prediction model, lubrication indicators may also be predicted and the lubrication indicator prediction result may be generated.

For example, in some embodiments, the lubrication health assessment result may be directly generated according to any one of the lubrication anomaly detection result, the lubrication failure mode classification result, the lubrication level classification result, and the lubrication indicator prediction result. For example, in some embodiments, the lubrication health assessment result may also be generated according to a combination of any one or more of the lubrication anomaly detection result, the lubrication failure mode classification result, the lubrication level classification result and the lubrication indicator prediction result. Thus, a lubrication health status of the target application and its relevant performance can be assessed from different perspectives such as lubrication anomaly degree, lubrication supply health degree, degradation degree, contamination degree, failure mode and severity and various lubrication indicators.

Based on the above disclosure, in the method for lubrication assessment of the present disclosure, multi-signal, multi-working condition, and multi-dimensional data related to lubrication are utilized and integrated, and thus more comprehensive relevant features can be extracted, so that a more effective correlation model between a target mechanical system (equipment or component) or process status to which lubrication is applied and a lubrication status can be established, so as to obtain more sensitive and accurate indicators, and finally can realize online and quantitative reflection and assessment of various situations and statuses about lubrication.

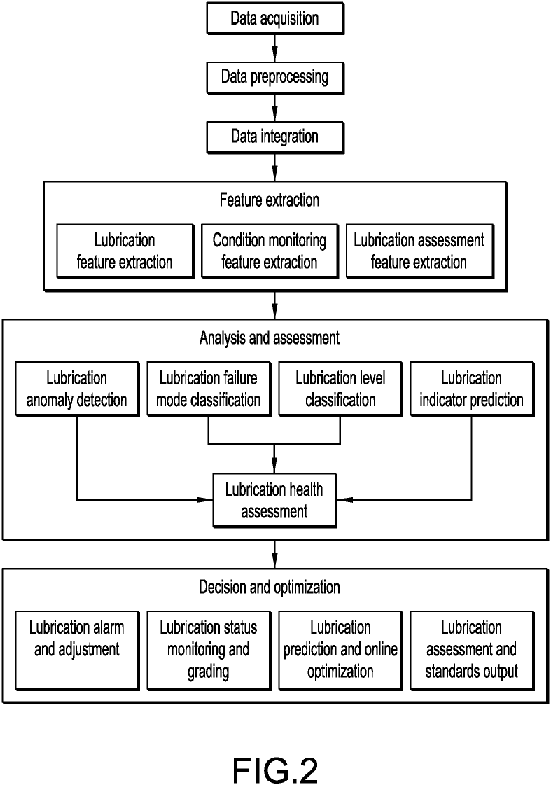

FIG. 2 illustrates a modular framework diagram of a method or system for lubrication assessment, decision and optimization according to one or more embodiments of the present disclosure. The modular framework diagram shown in FIG. 2 exemplarily shows a data acquisition module, a data preprocessing module, a data integration module, a feature extraction module, an analysis and assessment module, and a decision and optimization module. Each of the above modules may be implemented in software, in hardware, or in a combination of software and hardware.

The data acquisition module is intended to acquire required data. For example, the step at S101 in FIG. 1 may be performed to acquire working condition data related to the target lubrication, condition monitoring data related to the target lubrication, and lubrication assessment data related to the target lubrication.

The data preprocessing module is intended to process the acquired data. For example, the step at S102 in FIG. 1 may be performed to preprocess the acquired working condition data, condition monitoring data and lubrication assessment data to obtain preprocessed working condition data, preprocessed condition monitoring data and preprocessed lubrication assessment data.

The data integration module is intended to integrate the preprocessed data. For example, the step at S103 in FIG. 1 may be performed to perform data integration on the preprocessed working condition data, the preprocessed condition monitoring data and the preprocessed lubrication assessment data, to obtain the integrated data set.

The feature extraction module is intended to perform feature extraction on the integrated data set. For example, the step at S104 in FIG. 1 may be performed to perform feature extraction on data in the integrated data set according to data types and data characteristics based on the integrated data set to obtain the feature data set related to the target lubrication. As shown in FIG. 2, the working condition extraction module is intended to extract features of the working condition data based on the integrated data set to obtain working condition features. The condition monitoring feature extraction module is intended to extract features of the condition monitoring data based on the integrated data set to obtain condition monitoring features. The lubrication assessment feature extraction module is intended to extract features of the lubrication assessment historical data based on the integrated data set to obtain lubrication assessment features. Thus, based on the extracted working condition features, condition monitoring features, and lubrication assessment features, the target feature data set related to lubrication target may be constituted for subsequent modeling, analysis, and assessment.

The analysis and assessment module is intended to analyze and assess lubrication based on the extracted feature dataset. For example, steps at S105 and S106 in FIG. 1 may be performed to establish a lubrication analysis model for assessment of the target lubrication based on the feature data set; and assess the target lubrication and generate a lubrication assessment result based on the lubrication analysis model. For the analysis and assessment module exemplarily shown in FIG. 2, a lubrication anomaly detection module, a lubrication failure mode classification module, a lubrication level classification module and a lubrication indicator prediction module included therein may respectively establish corresponding analysis models as described above, generate corresponding analysis results according to outputs of corresponding analysis models, such as a lubrication anomaly detection result, a lubrication failure mode classification result, a lubrication level classification result, and a lubrication indicator prediction result. The lubrication health assessment module may generate a lubrication health assessment result reflecting lubrication from multiple perspectives and multiple dimensions, based on at least one of the lubrication anomaly detection result, the lubrication failure mode classification result, the lubrication level classification result, and the lubrication indicator prediction result.

The decision and optimization module in FIG. 2 is intended to make corresponding decisions and take relevant optimization actions based on the lubrication health assessment result. It should be appreciated that FIG. 2 only illustrates several main aspects, while in practical applications, selection and configuration may be made according to actual requirements. For example, a lubrication alarm is output based on the lubrication health assessment result, so an operator may take appropriate management and adjustment based on the lubrication alarm. For example, according to the lubrication health assessment result, monitoring situations and grading situations of lubrication status may be output online in various forms automatically or according to an operator’s retrieval. As a result, different optimization decisions and actions may be made according to different situations. For example, according to the lubrication health assessment result, lubrication may be optimized online accordingly based on a predicted trend of lubrication performance. For example, lubrication health results and standards may be generated and output according to actual requirements or the operator’s choice.

In addition, the lubrication assessment system of the present disclosure may further include a self-learning and improvement module. The self-learning and improvement module may, for example, continuously collect data, connect with an operating system and cooperate with production components, and update algorithms, logic and parameters based on a self-learning mechanism, thereby realizing automatic improvement of the system.

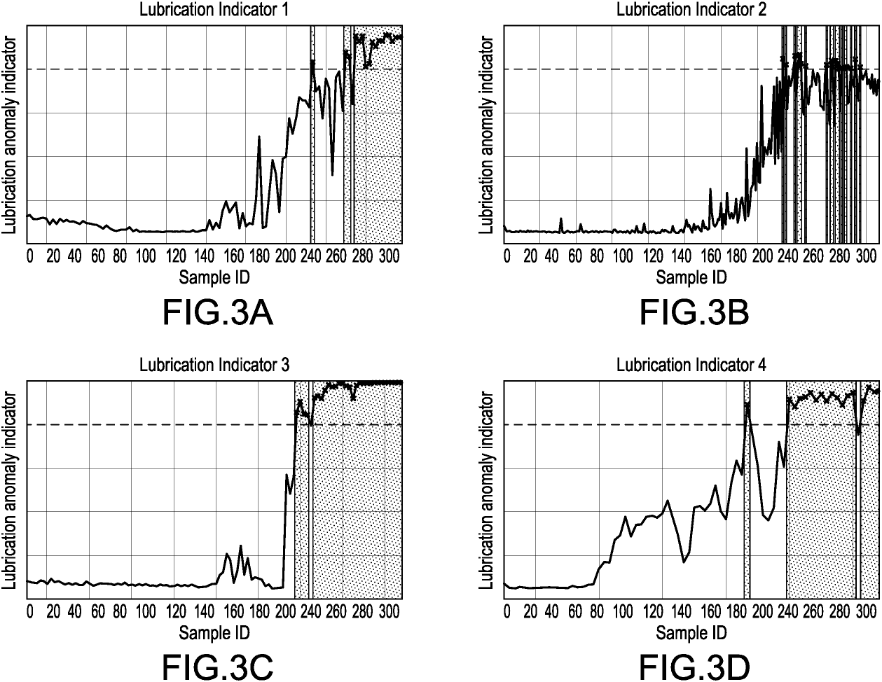

FIGS. 3A-3D illustrates an exemplary lubrication anomaly degree trend diagram according to the method and system of the present disclosure, which includes anomaly degree trend diagrams of four lubrication anomaly indicators. The lubrication anomaly indicators correspond to the outputs of the lubrication anomaly detection analysis model. Although the number of lubrication anomaly detection indicators output by the lubrication anomaly detection analysis model shown in this example is four, it should be appreciated that different lubrication anomaly detection analysis models may have different numbers of output indicators. In the example of FIGS. 3A-3D, data sampling is performed at a sampling frequency of 100 kHz, for example. In the four sub-graphs of FIGS. 3A-3D, the horizontal represents a sampling ID, the vertical axis represents a normalized value of a lubrication anomaly indicator, and the dotted line corresponds to a threshold for anomaly detection. In the figure, detected abnormal situations are prompted.

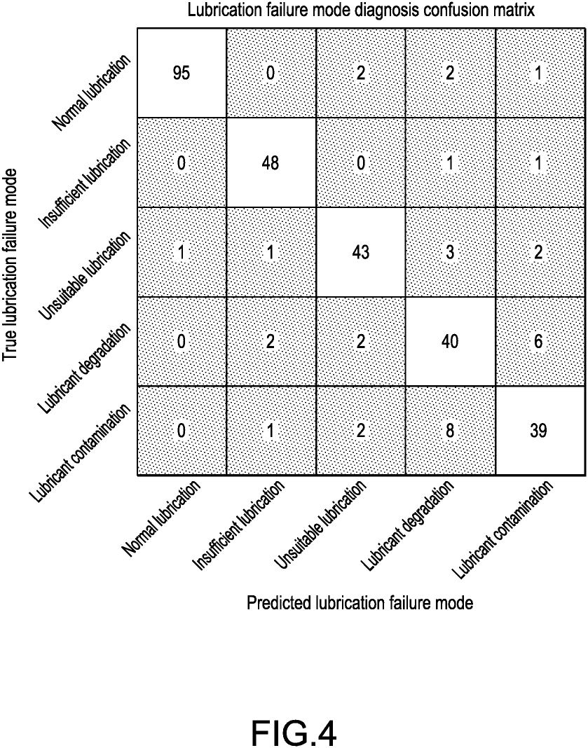

FIG. 4 illustrates a confusion matrix of lubrication failure mode classification results in a lubrication assessment process during testing. The predicted lubrication failure modes on the horizontal are classification results obtained by the lubrication failure classification model in the lubrication assessment method the system of the present disclosure, and the vertical axis represents true lubrication failure mode classification results. For example, the lubrication failure modes in this example are reflected in the following aspects: normal lubrication, insufficient lubrication, unsuitable lubricant, lubricant degradation, and lubricant contamination. It should be appreciated that other classifications may also be included as desired. This figure is for illustration only, and is not intended to limit the various embodiments of the present disclosure. Diagonal data of the matrix in the figure represents the number of samples with the same predicted results and true results. It can be seen that the lubrication assessment method and system of the present disclosure can establish an appropriate analysis model and generate analysis and assessment results with high accuracy.

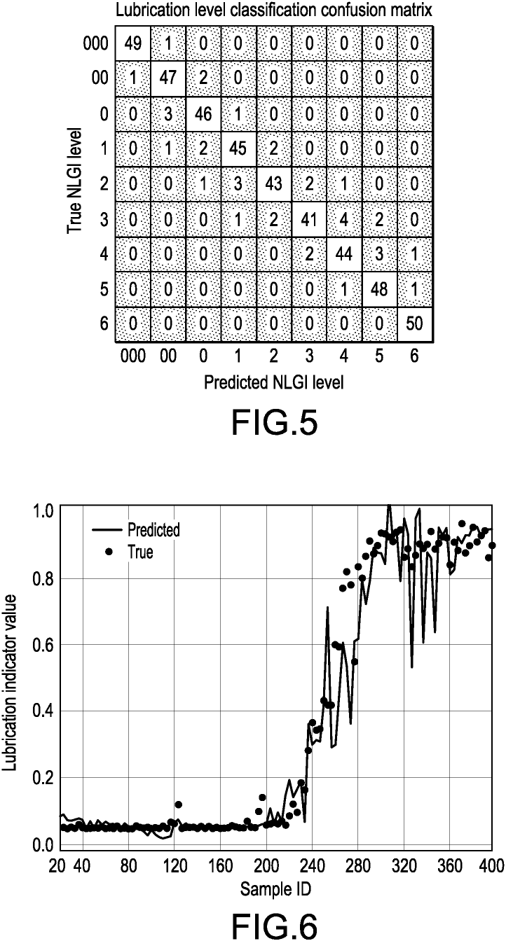

FIG. 5 illustrates a confusion matrix of lubrication level classification results in a lubrication assessment process during testing. The predicted level classifications (e.g., NLGI level) on the horizontal axis are results obtained by the lubrication level classification model in the lubrication assessment method and system of the present disclosure, and the vertical axis represents true lubrication level classification results. Diagonal data of the matrix in the figure represents the number of samples with the same predicted results and true results. It can be seen that the lubrication assessment method and system of the present disclosure can establish an appropriate analysis model and generate analysis and assessment results with high accuracy.

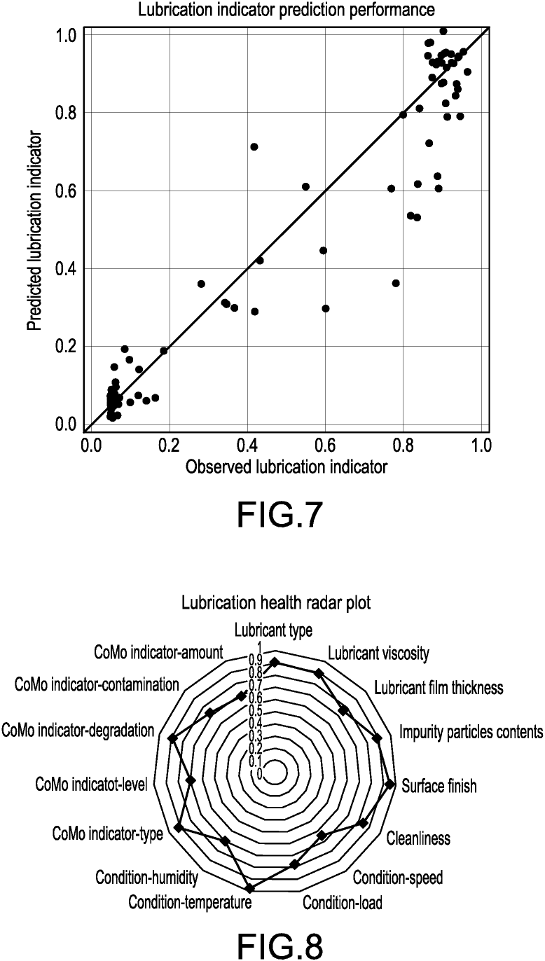

FIG. 6 exemplarily illustrates predicted results of a lubrication indicator during testing. In the example of FIG. 6, data sampling is performed at a sampling frequency of 100 kHz, for example. The horizontal axis indicates sampling IDs, and the vertical axis indicates normalized values of the lubrication indicator. The line represents fitting results of the lubrication indicator prediction obtained by the lubrication indicator prediction model in the lubrication assessment method and system of the present disclosure, and the circle dots represent true lubrication indicator results. It can also be seen from FIG. 6 that, by using the lubrication indicator prediction model in the method and the system for lubrication assessment of the present disclosure, the predicted lubrication indicators obtained according to the extracted features can well exhibit the lubrication status and trend. Thus, lubrication situations can also be predicted in advance using the lubrication assessment method of the present disclosure. It can be seen from the comparison that the lubrication assessment method and system of the present disclosure can establish an appropriate analysis model and generate analysis and assessment results with high accuracy. A fitting and regression performance of the lubrication indicator prediction is shown in FIG. 7.

FIG. 8 illustrates a radar chart of an exemplary lubrication health assessment based on partial health assessment results obtained using the lubrication assessment method and system of the present disclosure. In addition to conventional lubrication detection indicators (e.g., lubrication type, lubricating viscosity, lubricant film thickness, impurity particle content, surface finish, cleanliness), the radar chart further incorporates working condition parameters (e.g., speed, load, temperature, humidity), and lubrication health assessment indicators from model analysis (e.g., amount, contamination, degradation, level, type). It can be seen that, based on the comprehensive assessment of the lubrication assessment method and system of the present disclosure, more comprehensive, objective, quantitative and timely indicators can be obtained. These indicators can be used to assess, control and optimize lubrication, and even output more comprehensive standards for lubrication performance.

The lubrication assessment system of one or more embodiments of another aspect of the present disclosure may include a data collector and a processor coupled to the data collector. The data collector of the present disclosure may include various sensors with sensing functions, such as a speed sensor, a temperature sensor, a humidity sensor, and the like. The data collector may also include any device, interface or interface system connected to any system used for data collection and monitoring to obtain data from the system. The processor of the present disclosure may be a microprocessor, an Application Specific Integrated Circuit (ASIC), a System on a Chip (SoC), a computing device, a portable mobile computing device (e.g., a tablet or a cellphone), or the like. The processor may be configured to: preprocess the acquired working condition data, condition monitoring data, and lubrication assessment data to obtain preprocessed working condition data, preprocessed condition monitoring data, and preprocessed lubrication assessment data; perform data integration on the preprocessed working condition data, the preprocessed condition monitoring data, and the preprocessed lubrication assessment data to obtain an integrated data set; perform feature extraction on data in the integrated data set according to data types and data characteristics based on the integrated data set, to obtain a feature data set related to the target lubrication; establish a lubrication analysis model for assessment of the target lubrication based on the feature data set related to the target lubrication; and assess the target lubrication and generate a lubrication assessment result based on the lubrication analysis model.

The lubrication assessment method and system of the present disclosure can realize utilization and fusion of multi-signal, multi-working condition, and multi-dimensional data related to lubrication, extract more comprehensive features related to the lubrication, and therefore can establish more effective lubrication correlation models, thereby obtaining more sensitive and accurate indicators, to finally reflect and assess various conditions and statuses about the lubrication online and quantitatively.

Furthermore, the lubrication assessment method and system of the present disclosure can enrich and enhance existing lubrication inspection and assessment methods from non-invasive, timely, and quantitative perspectives, and thus can discover lubrication problems in time, classify and grade the lubrication failure modes and the severity online, predict the lubrication performance in advance, and optimize process parameters in real time. In addition, with the lubrication assessment method and system of the present disclosure, more indicators can be obtained for assessment and control of the lubrication performance objectively, quantitatively, and in time, and even more comprehensive lubrication performance standards can be output.

In addition, by use of the method and system of the present disclosure, it is possible to monitor, assess, control and optimize the lubrication process, status and performance in a continuous closed loop, thereby greatly enhancing the capability of lubrication assessment, the capability of control and the capability of providing solution. Furthermore, by processing and modeling acquired data based on large data or machine learning, it is possible to support assessment and optimization of lubrication in a digitalized and intelligent way.

Any one or more of the processor, memory, or system described herein include computer-executable instructions that can be compiled or interpreted from computer programs created using various programming languages and/or techniques. Generally, the processor, such as a microprocessor, receives and executes instructions, for example, from a memory, a computer-readable medium, or the like. The processor includes a non-transitory computer-readable storage medium capable of executing instructions of a software program. The computer-readable medium may be, but is not limited to, an electronic storage device, a magnetic storage device, an optical storage device, an electromagnetic storage device, a semiconductor storage device, or any suitable combination thereof.

The description of the embodiments has been presented for the purposes of illustration and description. Appropriate modifications and variations of the embodiments may be carried out in light of the above description or may be obtained by practice. For example, unless otherwise indicated, one or more of the methods described may be performed by a combination of suitable devices and/or systems. The method may be performed by utilizing one or more logic devices (e.g., processors) in conjunction with one or more additional hardware elements (such as storage devices, memories, circuits, hardware network interfaces, etc.) to perform stored instructions. The method and associated actions may also be performed in parallel and/or concurrently in various orders other than those described in this disclosure. The system described is exemplary in nature and may include additional elements and/or omit elements. The subject matter of the present disclosure includes all novel and non-obvious combinations of the various disclosed method and system configurations, and other features, functions, and/or properties.

As used in the present disclosure, an element or step listed in the singular and preceded by the word “a/an” should be understood as not excluding a plurality of said elements or steps, unless such exclusion is indicated. Furthermore, references to “one embodiment” or “one example” of the present disclosure are not intended to be interpreted as excluding existence of additional embodiments that also incorporate the recited features. The present disclosure has been described above with reference to specific embodiments. However, one of ordinary skill in the art will appreciate that various modifications and changes can be made without departing from the broader spirit and scope of the present disclosure as set forth in the appended claims.

What is claimed is:

1. A lubrication assessment method comprising:
acquiring working condition data related to target lubrication, condition monitoring data related to the target lubrication, and lubrication assessment data related to the target lubrication;
preprocessing the acquired working condition data, condition monitoring data, and lubrication assessment data to obtain preprocessed working condition data, preprocessed condition monitoring data, and preprocessed lubrication assessment data;
performing data integration on the preprocessed working condition data, the preprocessed condition monitoring data, and the preprocessed lubrication assessment data to obtain an integrated data set;
performing feature extraction on data in the integrated data set according to data types and data characteristics based on the integrated data set to obtain a feature data set related to the target lubrication;
establishing a lubrication analysis model for assessment of the target lubrication based on the feature data set related to the target lubrication; and
assessing the target lubrication and generating a lubrication assessment result, based on the lubrication analysis model.

2. The lubrication assessment method according to claim 1, wherein the performing feature extraction on data in the integrated data set according to the data types and the data characteristics based on the integrated data set to obtain the feature data set related to the target lubrication comprises:
extracting features of the working condition data based on the integrated data set to obtain working condition features;
extracting features of the condition monitoring data based on the integrated data set to obtain condition monitoring features;
extracting features of the lubrication assessment data based on the integrated data set to obtain lubrication assessment features;
obtaining the feature data set related to the target lubrication based on the working condition features, the condition monitoring features, and the lubrication assessment features.

3. The lubrication assessment method according to claim 2, wherein the obtaining the feature data set related to the target lubrication based on the working condition features, the condition monitoring features, and the lubrication assessment features comprises:
obtaining fused feature data by a feature fusion processing based on the working condition features, the condition monitoring features, and the lubrication assessment features, and generating the feature data set related to the target lubrication based on the fused feature data.

4. The lubrication assessment method according to claim 1, wherein the establishing the lubrication analysis model for assessment of the target lubrication based on the feature data set related to the target lubrication comprises:
establishing a lubrication anomaly detection model for detecting lubrication anomaly based on the feature data set related to lubrication;
establishing a lubrication failure mode classification model for classifying lubrication failure modes based on the feature data set related to lubrication;
establishing a lubrication level classification model for classifying lubrication levels based on the feature data set related to lubrication; and
establishing a lubricating indicator prediction model for predicting lubricating indicators based on the feature data set related to lubrication.

5. The lubrication assessment method according to claim 4, wherein the assessing the lubrication and generating the lubrication assessment result based on the lubrication analysis model comprises:
detecting lubrication anomalies and generating a lubrication anomaly detection result, based on outputs of the lubrication anomaly detection model;
classifying lubrication failure modes and generating a lubrication failure mode classification result, based on outputs of the lubrication failure mode classification model;
classifying lubrication levels and generating a lubrication level classification result, based on outputs of the lubrication level classification model;
predicting lubrication indicators and generating a lubrication indicator prediction result, based on the lubrication indicator prediction model; and
generating a lubrication health assessment result based on at least one of the lubrication anomaly detection result, the lubrication failure mode classification result, the lubrication level classification result, and the lubrication indicator prediction result.

6. The lubrication assessment method of claim 1, wherein the preprocessing the acquired working condition data, condition monitoring data, and lubrication assessment data comprises performing at least one of data deduplication processing, data denoising processing, data encoding processing and data filtering processing.

7. The lubrication assessment method according to claim 1, wherein the performing data integration on the preprocessed working condition data, the preprocessed condition monitoring data, and the preprocessed lubrication assessment data comprises:
performing at least one of synchronization, alignment, and correction processing on the preprocessed working condition data, the preprocessed condition monitoring data, and the preprocessed lubrication assessment data.

8. The lubrication assessment method according to claim 1, further comprising optimizing the target lubrication based on the lubrication assessment result.

9. A lubrication assessment system comprising:
a data collector configured to acquire working condition data related to target lubrication, condition monitoring data related to the target lubrication, and lubrication assessment data related to the target lubrication; and
a processor connected to the data collector configured to:
preprocess the acquired working condition data, condition monitoring data, and lubrication assessment data to obtain preprocessed working condition data, preprocessed condition monitoring data, and preprocessed lubrication assessment data;
perform data integration on the preprocessed working condition data, the preprocessed condition monitoring data, and the preprocessed lubrication assessment data to obtain an integrated data set;
perform feature extraction on data in the integrated data set according to data types and data characteristics based on the integrated data set to obtain a feature data set related to the target lubrication;
establish a lubrication analysis model for assessment of the target lubrication based on the feature data set related to the target lubrication; and
assess the target lubrication and generate a lubrication assessment result, based on the lubrication analysis model.

10. A non-transient computer-readable storage medium having computer-readable instructions stored thereon, wherein when the instructions are executed by a computer, the method of claim 1 is performed.

11. A non-transient computer-readable storage medium having computer-readable instructions stored thereon, wherein when the instructions are executed by a computer, the method of claim 5 is performed.
